(19) United States
(12) Patent Application Publication
(10) Pub. No.: US 2022/0117524 A1
(43) Pub. Date: Apr. 21, 2022

(54) APPARATUS, METHOD AND DEVICE FOR NON-CONTACT AND NON-INVASIVE BLOOD SUGAR MONITORING TO HELP MONITOR DIABETIC PATIENTS AND HYPERCOAGULATION

(71) Applicants: Pai-chang Yeh, Zhubei (TW); Julian Gerald Dcruz, KL (IN)

(72) Inventors: Pai-chang Yeh, Zhubei (TW); Julian Gerald Dcruz, KL (IN)

(21) Appl. No.: 17/645,984

(22) Filed: Dec. 25, 2021

(51) Int. Cl. A61B 5/145; A61B 5/00

(52) U.S. Cl. CPC A61B 5/14532; A61B 5/0077; A61B 5/7264; A61B 5/0066

(57) ABSTRACT

The present invention relates to apparatus, method and a device for non-contact & non-invasive blood sugar monitoring blood sugar and related diseases caused due to variations in blood sugar. The apparatus includes a camera to obtain a real-time video of the user. Such real-time video can be forwarded to a processor and an AI database via a platform. The processor is configured to process at least each frame from the obtained real-time video; extract one or more facial regions from the each of the processed frames to thereby extract one or more regions of interest present therein; and feed the one or more extracted regions of interest to at least one image based physiological monitoring model along with one or more Photoplethysmography imaging (iPPG) and Optical Coherence Tomography variations to process the one or more extracted regions of interest and obtain at least one result indicative of the blood sugar level of the of user based on the real-time video by using Convolutional Neural Network algorithm.

### TECHNICAL FIELD

The present disclosure relates generally to monitoring the physiological conditions of one or more individuals in an unobtrusive ongoing manner, by using images acquired by one or more digital capture devices. More particularly, it relates to apparatus, method and a device for non-contact and non-invasive blood sugar monitoring blood sugar and related diseases caused due to variations in blood sugar.

### BACKGROUND

Background description includes information that may be useful in understanding the present invention. It is not an admission that any of the information provided herein is prior art or relevant to the presently claimed invention, or that any publication specifically or implicitly referenced is prior art. The following description includes information that may be useful in understanding the present invention. It is not an admission that any of the information provided herein is prior art or relevant to the presently claimed invention, or that any publication specifically or implicitly referenced is prior art.

Blood sugar refers to the sugar found in our blood, it comes from the food we eat and provides the main source of energy required for our body to function, the blood carries the glucose to all the body cells to produce the required energy. Diabetes is a disease in which your blood sugar levels are too high. Over time, having too much glucose in your blood can cause serious problems. Even if you don't have diabetes, sometimes you may have problems with blood sugar that is too low or too high. Keeping a regular schedule of eating, activity, and taking any medicines you need can help. If you do have diabetes, it is very important to keep your blood sugar numbers in your target range. You may need to check your blood sugar several times each day. Your health care provider will also do a blood test called an HbA1C. It checks your average blood sugar level over the past three months. If your blood sugar is too high, you may need to take medicines and/or follow a special diet.

Below we have some tables which show the ranges of Blood sugar levels (NICE recommended target blood glucose level ranges):

| | | | At least 90 minutes |
|---|---|---|---|
| Target Levels by | | Before meals (pre- | after meals (post |
| Type | Upon Waking | prandial) | prandial) |
| Non-Diabetic | | 4.0 to 5.9 mmol/L | Under 7.8 mmol/L |
| Type 2 Diabetes | | 4 to 7 mmol/L | Under 8.5 mmol/L |
| Type 1 Diabetes | 5 to 7 mmol/L | 4 to 7 mmol/L | 5 to 9 mmol/L |
| Children w/type 1 | 4 to 7 mmol/L | 4 to 7 mmol/L | 5 to 9 mmol/L |
| diabetes | | | |

Blood sugar levels in diagnosing diabetes:

| Plasma Glucose Test | Normal | Prediabetes | Diabetes |
|---|---|---|---|
| Random | Below 11.1 mmol/L | N/A | 11.1 mmol/L or |
| | | | More |
| Fasting | Below 5.5 mmol/L | 5.5 to 6.9 mmol/L | 7.0 mmol/L or more |
| 2 hour post prandial | Below 7.8 mmol/L | 7.8 to 11.0 mmol/L | 11.1 mmol/L or |
| | | | more |

Blood sugar levels give you information about how well your diabetes is under control. They also tell you how well your plan of diet, exercise, and medicine is working. Keeping your blood sugar levels near normal may reduce or prevent your risk for problems (complications).

The devices used by health care professionals to monitor blood sugar levels include Digital Glucometers, which involve finger pricking the user to test the glucose levels in the blood, the health care professionals can also instruct the user to take a Fasting or Random plasma test or Oral glucose tolerance test or even a HbA1c test for diagnosing diabetes. These devices involve patient contact with the device which can be categorised as invasive or in-contact.

Monitoring Blood sugar levels continuously can help health care professionals and users to take precaution in their health accordingly, this is especially applicable for patients having cardio-vascular problems.

A prior-art reference U.S. Pat. No. 6,611,206 by Eshelman et al., and U.S. Pat. No. 6,968,294 by Gutta et al., anticipate the need for home health monitoring of individuals, such as the elderly, who would normally need a caretaker to protect their health. The monitoring systems of these patents includes a pervasive array of sensors, including cameras, to enable monitoring of the subject relative to behavior, emotional state, activity, safety, environment, and security. These systems also include devices to provide local or remote alerts concerning the subject and his or her environment. The systems of Eshelman '206 and

Gutta '294 are neither unobtrusive nor intended for generalized family health care. Additionally, these systems really do not provide imaging-based health assessments for multiple individuals that address the variability that would be expected, including variations in age, ethnicity, ambient lighting conditions, seasonally induced changes in appearance, privacy, health history, and other factors.

Another patent, U.S. Pat. No. 6,539,281 by Wan et al., provides for a medicine cabinet or similar device that assists users in selecting, taking, and tracking their use of medications. In this instance, the medications are provided with radio frequency identification tags, and the medicine cabinet is equipped with a radio frequency tag reader. A touch screen flat panel display can be provided with the cabinet, as an interface to the users.

The cabinet may include a camera and face recognition software, to enable user identification. While the intelligent medicine cabinet of Wan '281 is useful, it does not use a camera for assessing the physiological state or conditions of the users, and as such, it does not anticipate either the issues or opportunities that arise from such considerations.

There are other health care devices that are more focused on the home monitoring of health or medical parameters, rather than general behavior and activity. As an example, international patent publication W02001/071636 by O'Young describes a personalized health profiling system intended to collect quantitative health data on individuals in their home environments, so as to look for warning signs of potential disease or a changes in one's health or physical state. The data collection is intended to be sufficiently unobtrusive that it can be undertaken during normal daily activities, such as working, sleeping, or exercising. In particular, O'Young '636 anticipates that one or more sensors are to be worn by an individual proximate to their body, to monitor heart rate, blood oxygenation, gait rhythm, or body temperature. Similarly, international patent publication WO2005/006969 by Montvay et al. anticipates a health monitoring system that enables health related-coaching of an individual who may be in their own home. This system can have sensors that are worn by an individual, or implanted in their body. Such sensors can monitor the electrocardiogram (ECG) or a respiration rate of the individual. Other sensors can be provided, for example mounted to a wall, to monitor environmental data, like air temperature, humidity, and other parameters. While the devices and systems of O'Young '636 and Montvay '969 are targeted for home health care, they are not targeted for generalized family health care. In particular, they do not anticipate an unobtrusive system capable of ongoing, day after day, monitoring of multiple individuals. Additionally, none of these systems provides image normalization to account for the variability associated with multiple individuals, lighting conditions, seasonal changes, and other factors.

Thus, there is a need for users to have easy access to monitor blood sugar levels with ease and easy accessibility.

### SUMMARY

The present disclosure relates generally to monitoring the physiological conditions of one or more individuals in an unobtrusive ongoing manner, by using images acquired by one or more digital capture devices. More particularly, it relates to apparatus, method and a device for non-contact and non-invasive blood sugar monitoring blood sugar and related diseases caused due to variations in blood sugar.

The invention presented here provides an apparatus and a method for the non-contact and non-invasive monitoring of blood sugar.

The solution is developed across multiple platforms for the ease of access and use for users. The solution consists of the Docsun Health Monitoring Web and Mobile Application, also another solution involves Docsun DSXXXX Series Doorway Terminal Devices (For the Purpose of Public places).

The requirements for the software to analyse and give proper monitoring of Blood sugar is that the device running the software requires good high-quality camera and the user should be seen in well-lit environment with sufficient light on the face. The user should refrain from using any type of eye-lens, make-up and mask objects that cover his/her facial regions.

The invention provides:
a) Simple and Easy to access interface for User to Measure Blood sugar in a non-contact method from his phone or in a public place.
b) Docsun Device Monitor can be setup to monitor blood sugar levels continuously for users.
c) Cost-effective solution compared to medical devices.
d) Utilises State of the Art Technology AI to power the device.

### BRIEF DESCRIPTION OF THE DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates exemplary block diagram of apparatus for non-contact and non-invasive monitoring of blood sugar level of a user, in accordance with an exemplary embodiment of the present disclosure.

FIGS. 2A-2B illustrates an exemplary waveform indicative of a readings taken by the apparatus as blood sugar level predicted and blood sugar levels obtained in real-time, in accordance with an exemplary embodiment of the present disclosure.

FIG. 3 illustrates a perspective of a user of the present invention interacting with a system capable of providing the present invention, in accordance with an exemplary embodiment of the present disclosure.

FIG. 4 illustrates depicting the present invention configured as a network, in accordance with an exemplary embodiment of the present disclosure.

FIG. 5 illustrates non-contact and non-invasive method for monitoring of blood sugar level of a user, in accordance with an exemplary embodiment of the present disclosure.

FIG. 6 illustrates exemplary physical components of an apparatus or a device for non-contact and non-invasive monitoring of blood sugar level of a user, in accordance with an exemplary embodiment of the present disclosure.

### DETAILED DESCRIPTION

The following detailed description is made with reference to the technology disclosed. Preferred implementations are described to illustrate the technology disclosed, not to limit its scope, which is defined by the claims. Those of ordinary skill in the art will recognize a variety of equivalent variations on the description.

Examples of systems, apparatus, computer-readable storage media, and methods according to the disclosed implementations are described in this section. These examples are being provided solely to add context and aid in the understanding of the disclosed implementations. It will thus be apparent to one skilled in the art that the disclosed implementations may be practiced without some or all of the specific details provided. In other instances, certain process or method operations also referred to herein as “blocks,” have not been described in detail in order to avoid unnecessarily obscuring the disclosed implementations. Other implementations and applications also are possible, and as such, the following examples should not be taken as definitive or limiting either in scope or setting.

In the following detailed description, references are made to the accompanying drawings, which form a part of the description and in which are shown, by way of illustration, specific implementations. Although these disclosed implementations are described in sufficient detail to enable one skilled in the art to practice the implementations, it is to be understood that these examples are not limiting, such that other implementations may be used and changes may be made to the disclosed implementations without departing from their spirit and scope. For example, the blocks of the methods shown and described herein are not necessarily performed in the order indicated in some other implementations. Additionally, in some other implementations, the disclosed methods may include more or fewer blocks than are described. As another example, some blocks described herein as separate blocks may be combined in some other implementations. Conversely, what may be described herein as a single block may be implemented in multiple blocks in some other implementations. Additionally, the conjunction “or” is intended herein in the inclusive sense where appropriate unless otherwise indicated; that is, the phrase “A, B or C” is intended to include the possibilities of “A,” “B,” “C,” “A and B,” “B and C,” “A and C” and “A, B and C.”

Some implementations described and referenced herein are directed to systems, apparatus, computer-implemented methods and computer-readable storage media for detecting flooding of message queues.

Thus, for example, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any electronic code generator shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular named.

Various terms as used herein are shown below. To the extent a term used in a claim is not defined below, it should be given the broadest definition persons in the pertinent art have given that term as reflected in printed publications and issued patents at the time of filing.

The present disclosure relates generally to monitoring the physiological conditions of one or more individuals in an unobtrusive ongoing manner, by using images acquired by one or more digital capture devices. More particularly, it relates to apparatus, method and a device for non-contact and non-invasive blood sugar monitoring blood sugar and related diseases caused due to variations in blood sugar.

The present invention relates to apparatus, method and a device for non-contact & non-invasive blood sugar monitoring blood sugar and related diseases caused due to variations in blood sugar. The apparatus includes a camera to obtain a real-time video of the user. Such real-time video can be forwarded to a processor and an AI database via a platform. The processor is configured to process at least each frame from the obtained real-time video to obtain at least one result indicative of the blood sugar level of the of user and provide warning related to diseases caused due to variations in blood sugar.

The Invention is developed in C-Language, C++ Language, java-script and python utilising capabilities for multiple platform developments on web, android, iOS, windowsX86 and linuxX86 based devices. The software requires the use of an optical camera.

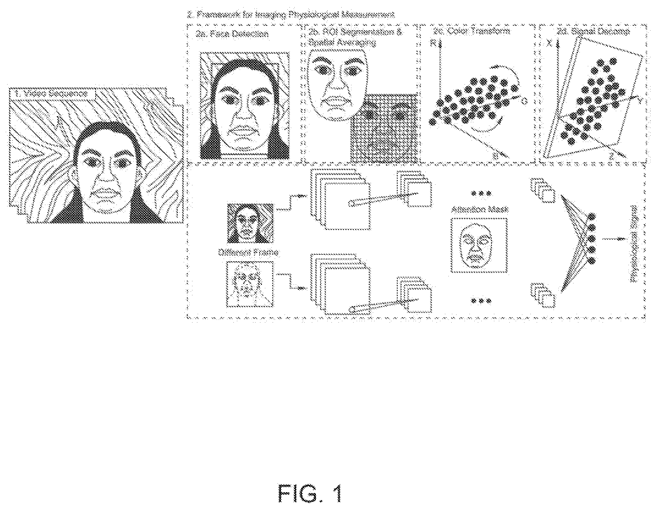

FIG. 1 illustrates exemplary block diagram of apparatus for non-contact and non-invasive monitoring of blood sugar level of a user, in accordance with an exemplary embodiment of the present disclosure.

As shown in FIG. 1, the Software processes real-time video, each frame of the video is processed with de-noising profiles after which we run few layers of varying augmentation profiles. The Facial regions are extracted, and the regions of interest are extracted and filtered before providing it as input to the model. We also supply the iPPG and Optical Coherence Tomography (OCT) variations into the model as input to add as Correlation labels by using Convolutional Neural Network Algorithm (CNN). These serve to help better the results and add a sense of fine tuning to the readings. The result is then categorised as 3 different alerts—Green Alert (For Healthy), Yellow Alert (Caution) and Red Alert (Abnormal). These alerts provide a sense of understanding and ease to use interface to the user. The result shown to user consists of blood sugar level readings in mmol/L and mg/dL and also health alert with textual wordings to help user understand the alert.

The Model processes the facial regions and extracts multi corelating regions and we have added an additional filter profile to help bring corelation to the extracted regions before being sent to the model, this brings consistency in predictions and helps improve accuracy.

The alert wordings are: Normal Blood Sugar Level, and Abnormal Blood Sugar Level. The numerical readings are represented as mmol/L and mg/dL readings.

The readings are generated within 30 seconds and provide time-efficient readings and can also be used in a continuous readings state for continuous monitoring which is helpful for patients suffering from diabetes, kidney-related diseases, angina, hypertension, etc. to monitor their blood sugar readings in a continuous non-contact and non-invasive way.

A benchmark study was conducted in Kenya and a Laboratory study was conducted in Taiwan to validate the bias, and accuracy for the blood sugar predictions. The study results showed no substantial variations in bias for populations of different racial origin and skin colour. The study was conducted, and readings were validated using a standard digital glucometer, the readings from the digital glucometer and the Docsun health monitoring software was compared to calculate the accuracy and the bias.

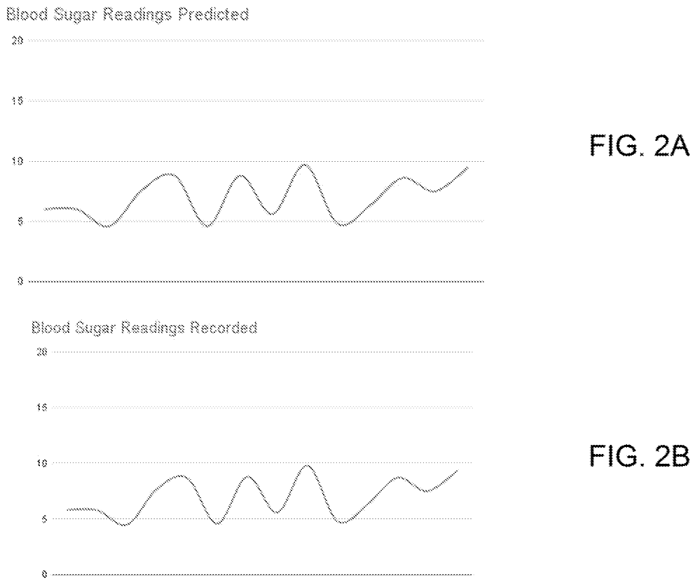

The Bias for the readings is: less than 0.5 mmol/L. The Bias was calculated after considering the bias for the device used to validate the accuracy. The Device gave accurate predictions of blood sugar with slight variations, this was identified as artifact noise due to motion and light. The inclusion for factoring into noise from motion and light artifacts are provided as calibration to help maintain the bias and improve readings accuracy. FIGS. 2A-2B illustrates an exemplary waveform indicative of a readings taken by the apparatus as blood sugar level predicted and blood sugar levels obtained in real-time, in accordance with an exemplary embodiment of the present disclosure. The model performed with 98% accuracy with 2% error rate, the graphical plot depicting the study readings for users of different racial origin and skin colour.

The known and potential benefit of the Self Diagnostic AI Software for clinical use of screening and diagnosis of covid19 are: fast and accurate readings of Blood sugar levels, which are then processed to identify the user's symptoms and detect if the person is suffering from hypertension or healthy, quality Assured user experience of the application, and protection of user privacy.

As used herein, the term engine refers to software, firmware, hardware, or other component that can be used to effectuate a purpose. The engine will typically include software instructions that are stored in non-volatile memory (also referred to as secondary memory). When the software instructions are executed, at least a subset of the software instructions can be loaded into memory (also referred to as primary memory) by a processor. The processor then executes the software instructions in memory. The processor may be a shared processor, a dedicated processor, or a combination of shared or dedicated processors. A typical program will include calls to hardware components (such as I/O devices), which typically requires the execution of drivers. The drivers may or may not be considered part of the engine, but the distinction is not critical.

As used herein, the term database is used broadly to include any known or convenient means for storing data, whether centralized or distributed, relational or otherwise.

Embodiments of the present invention include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and firmware and/or by human operators.

Embodiments of the present invention may be provided as a computer program product, which may include a machine-readable storage medium tangibly embodying thereon instructions, which may be used to program a computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other type of media/machine-readable medium suitable for storing electronic instructions (e.g., computer programming code, such as software or firmware).

Various methods described herein may be practiced by combining one or more machine-readable storage media containing the code according to the present invention with appropriate standard computer hardware to execute the code contained therein. An apparatus for practicing various embodiments of the present invention may involve one or more computers (or one or more processors within a single computer) and storage systems containing or having network access to computer program(s) coded in accordance with various methods described herein, and the method steps of the invention could be accomplished by modules, routines, subroutines, or subparts of a computer program product

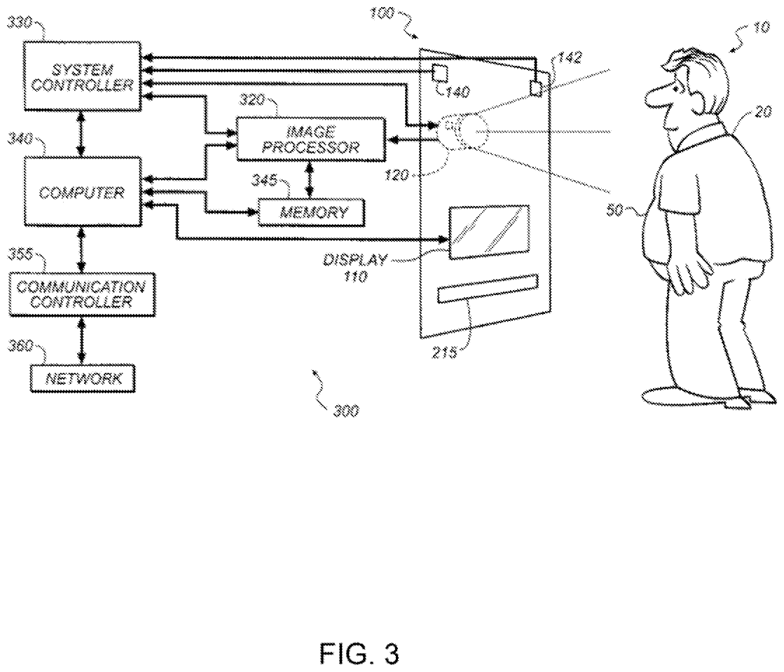

FIG. 3 illustrates a perspective of a user of the present invention interacting with a system capable of providing the present invention, in accordance with an exemplary embodiment of the present disclosure.

In a broader context, the hardware for blood sugar level monitoring system 300. The primary elements of blood sugar level monitoring system 300 are the electronic imaging device 100, which includes at least one camera 120, and possibly a display 110. Electronic imaging device 100 is interconnected to image processing electronics 320, a system controller 330, a computer 340, memory or data storage 345, a communications controller 355, and a network 360. The image processing electronics 320 potentially serve multiple purposes, including improving the quality of image capture of the camera 120 associated with a local electronic imaging device 100, improving the quality of images displayed at a local display 110, and processing the captured images to aid the derivation of metrics relative to physiological conditions. Computer 340 coordinates control of the image processing electronics 320 and system controller 330. Computer 340 also manipulates and accesses data from memory 345, display 110, image processing electronics 320, and network 360. Both image processing electronics 320 and computer 340 can access various databases (which will be discussed subsequently), many of which are stored in memory 345. System controller provides various control and driver functions for a local electronic imaging device 100, including display driver and image capture control functions. A variety of detectors can be provided, including an ambient light detector 140, a motion detector 142, and various secondary detectors 144 that can be used for measuring ambient light or other physiological or environmental parameters. These detectors are interconnected with computer 340 or controller 330. Communication controller 355 acts as interface to a communication channel, such as a wireless or wired network 360, for transferring image and other data from one site to the other.

As noted previously, the principal anticipated application of blood sugar level monitoring system 300 is in the residential market. Yet unfulfilled needs can be identified from a purely medical perspective and from a broader context of human well-being and health. The newspaper, USA Today, reports that the United States could have a shortage of 85,000 to 200,000 doctors in 2020, fuelled not only by malpractice insurance and other non-medical business issues impacting the numbers of students who go into medicine, but also by 79 million baby boomers reaching retirement age and needing more medical care. Further, decreasing contributions towards health care from employers and governmental entities will mean that consumers will pay much more for health care. These pressures will likely force increasing health care expenses upon consumers, which might be somewhat ameliorated if consumers can better assess if and when intervention by health care professionals is warranted.

Undeniably, the doctor shortages, the increased needs of baby boomers, and the diminishing contributions made by innumerable employer's means that consumers increasingly have to take greater control over their own and their family's health care. Significant care for acute conditions will likely be shouldered by the “sandwich generation”, i.e., almost 3 in 10 of those aged 45 to 64 with children in the home, who are also caring for a senior, according to a study based on the 2002 General Social Survey. And many of those parents who do not have their own elderly parents living in their homes are still anxious about their elderly parent's health, especially when the elderly live in distant locales.

Although system 300 can obtain data on a daily basis, many wellness parameters (specifically bllo sugar level in the present invention) 410 will generally change very slowly, and thus some wellness data can be measured and retained on a less frequent basis. For example, as physical attributes such as weight or posture tend to change slowly, the associated wellness parameters (specifically bllo sugar level in the present invention) can be sought or retained on a weekly, monthly, or quarterly basis, depending on the attribute or trait in question and the variability associated with its measurement.

Thus, the system 300 is intended to enable the collection of a record of physiological data for one or more individuals. To enable this, the system 300 is provided with a dual-purpose device, and in particular an electronic imaging device 100 that unobtrusively captures images of a user or subject via one or more cameras 120. Electronic imaging device 100 can be a computer monitor, television, cell phone, mirror, or other display that sees the subject (with a camera 120) while the subject (user 10) is looking into the device. As shown in FIG. 3, electronic imaging device 100 is a computer, such as desktop or laptop system. The camera 120 can be mounted at the display edge (as shown), or be integrated into electronic imaging device 100, such that it looks through the display 110 at a user 10. Whereas, the electronic imaging device 100 includes a mirror 136 integrated with a camera 120 and (optionally) a display 110. A camera 120 typically includes an imaging lens 122 that provides an image onto an image sensor array 124, through a spectral filter 126. In this case, camera 120 can look through an aperture A, for example provided by a semi-transparent mirror 134. To aid in hiding the camera 120 and aperture A, semi-transparent mirror 134 can have a gradient reflectance, with the lowest reflectance in the centre of aperture A. The semi-transparent mirror 134 can also be a flickering device that is driven electronically to switch between reflecting and transmitting states. Alternately aperture A can be an optical pinhole (<0.5 mm diameter), making camera 120 a pinhole camera. In any case, cameras 120 are preferably hidden within device 100, and not generally visible to the users. As shown in FIG. 3, blood sugar level monitoring system 300 can be networked, and utilize several electronic imaging devices 100 within a residence, including both the computer monitor and mirror types. In principal, the intention is that the physiological images are unobtrusively collected while the subject or subjects look into the mirror or display, which they are already doing to view themselves, or to view information, communications, or entertainment. These captured images can be acquired day after day, month after month, and year after year, resulting in a rich image-based representation of the subjects over long periods of time.

Although the configuration of blood sugar level monitoring system 300 as a distributed network is particularly advantageous relative to capturing physiological image based data for multiple family members, various issues regarding individual and family privacy are accentuated. In particular, placement of electronic imaging devices 100 as one or more bathroom mirrors is advantageous relative to the image capturing. For example, in a household, a mirror type electronic imaging device 100 can be provided in the master bathroom, while another can be provided in a children's bathroom. Considering human behavioural patterns involving personal grooming, the best opportunity for capturing image data on a day after day basis could be from the mirror type electronic imaging devices 100.

Also, the most repeatable, and perhaps the best, set of illumination conditions might be found in the bathroom setting. However, as can then be anticipated, management of user privacy, particularly in the bathroom setting, is very important. On the other hand, electronic imaging devices 100 that are integrated into a computer, television, or entertainment station would be expected to see regular usage on a daily basis, or nearly so, depending on the household.

Although the privacy concerns related to image capture from these non-bathroom located devices might be reduced, the image capture conditions may be both inferior and more variable. In any case, various hardware and software design features can be integrated into blood sugar level monitoring system 300 to address privacy concerns and any associated variability inherent in the capture conditions.

Notably, it is not sufficient to simply capture an image, but image assessment, enabled by image normalization, is key. Again, considering a home environment, the appearance of family members can vary significantly relative to gender, age, skin color, hair color, height, weight, and other factors. Likewise, the basic appearance of any individual can vary by season (such as tanned or sun-burnt), by behavior (including use of cosmetics, exercise, or alcohol and drug use or abuse), and by other factors. The ambient lighting can also change dramatically from one image capture opportunity to the next. In a similar fashion, the position of an individual relative to the image capture device can lead to variation in the size, orientation, or placement of the individual in the captured image. Therefore, to compensate for these wide ranges of variables that can affect image capture and interpretation with unobtrusive image capture, the process of blood sugar level monitoring employs an image normalization process to decrease the impact of the capture variables. In particular, the capture step is followed by the image normalization process, which modifies the captured imagery before size or color-based wellness parameters (specifically bllo sugar level in the present invention) are derived from the image data. Processes for assessing physiological conditions of the subjects then follow the data normalization process. Likewise, these processes for assessing or inferring a subject's well-being must account for subject variability relative to appearance, behavior, privacy, and other factors.

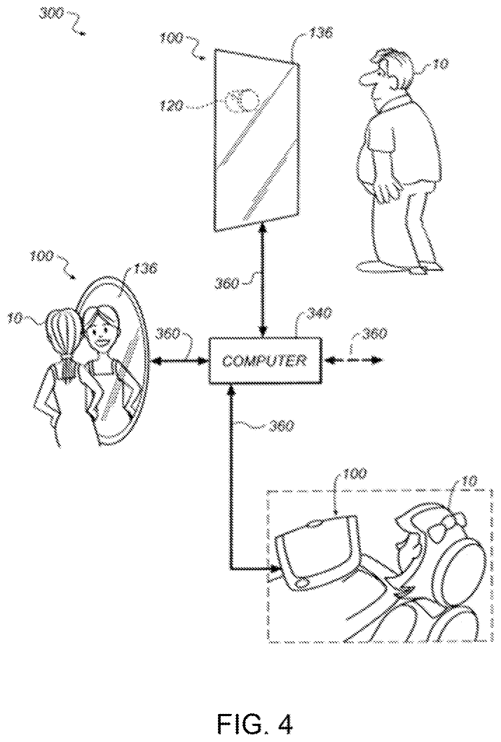

FIG. 4 illustrates depicting the present invention configured as a network, in accordance with an exemplary embodiment of the present disclosure.

Although the blood sugar level monitoring system 300 has been described, relative to FIG. 4, as a networked system, the system had been predominately described as including an electronic imaging device 100 built into a bathroom vanity. Application in that environment can impose particular limitations. For example, the ability of the electronic imaging device 100 to capture images can be impaired if the mirror 136 is fogged by condensation as might occur when an individual takes a shower. Of course, the outer, mirrored surface can be heated or coated to reduce this problem. It is also recognized that in many bathrooms, medicine cabinets are provided behind the mirror. Certainly, it can be expected that any cameras 120, displays 100, or other detectors or sensors (136, 142, 144) will be competing for at least some of this potential space. Again considering FIG. 3, electronic imaging devices 100 for physiological monitoring system 300 can be positioned elsewhere within a residence, including behind pictures or bedroom vanities. As another example, one or more cameras 120 can be positioned at a computer or a television, much like how web-cameras are used today. In such cases, the physiological monitoring system 300 can observe other factors than the wellness parameters 410 previously described. For example, the physiological monitoring system 300 can observe the posture, ergonomics, emotional response, attention span, time spent, and fatigue of a subject 10 at a computer or television.

Such data can be useful in assessing mental stress levels, potential repetitive stress disorders such as carpal tunnel syndrome, or mental attention to a task. In the case of children, assessments of mental attention can be useful relative to conditions like attention deficit disorder (ADD) or for understanding educational performance. Additionally, the physiological monitoring system 300 can also accept inputs from other bio-medical devices, including hand held or wearable sensors. These supplemental devices can also include PDA or cell phone type devices that have imaging capabilities.

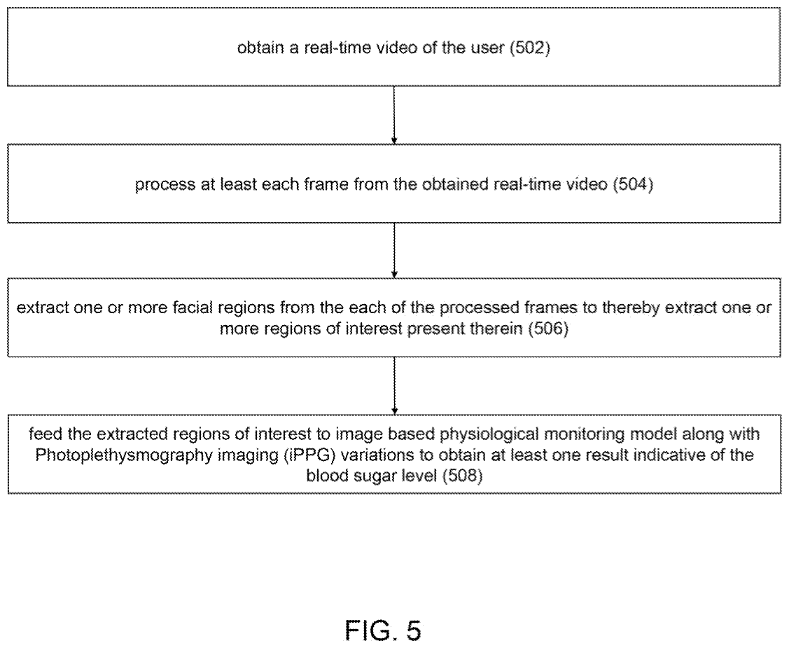

FIG. 5 illustrates non-contact and non-invasive method for monitoring of blood sugar level of a user, in accordance with an exemplary embodiment of the present disclosure.

In an exemplary embodiment, a non-contact and non-invasive method for monitoring of blood sugar level of a user is disclosed. The non-contact and non-invasive method includes the steps of:

At step 502, one or more cameras obtain a real-time video of the user

At step 504, a processor processes at least each frame from the obtained real-time video.

At step 506, the processor extracts one or more facial regions from the each of the processed frames to thereby extract one or more regions of interest present therein.

At step 508, the processor feeds the one or more extracted regions of interest to at least one image based physiological monitoring model along with one or more Photo plethysmography imaging (iPPG) and Optical Coherence Tomography (OCT) variations to process the one or more extracted regions of interest by using Convolutional Neural Network (CNN) algorithm and obtain at least one result indicative of the blood sugar level of the of user based on the real-time video.

The at least one obtained result provides the blood sugar level indication in at least one of a healthy range, a caution range, and an abnormal range category. Alternatively, he at least one obtained result provides an indication of one or more possible predicted diseases based on the at least one obtained result.

In an exemplary embodiment, the at least one image based physiological monitoring model.

In an exemplary embodiment, the one or more Photo plethysmography imaging (iPPG) and Optical Coherence Tomography (OCT) variations are feed to the at least one image based physiological monitoring model to add one or more correlation labels while obtaining the at least one result.

In an exemplary embodiment, the at least one image based physiological monitoring model utilizes an artificial intelligence (AI) or deep learning techniques or a trained classifier to obtain the at least one result.

In an exemplary embodiment, the at least one image based physiological monitoring model comprise of Convolutional Neural Network (CNN) algorithm or a software to obtain the at least one result.

In an exemplary embodiment, the step of processing further comprising de-noising profiles and executing one or more augmentation on the denoised profiles

In an exemplary embodiment, the step of extracting further comprising filtering the one or more regions of interest before providing to at least one image based physiological monitoring model.

In an exemplary embodiment, the step of processing the one or more extracted regions of interest by the at least one image based physiological monitoring model further comprising extracting multi corelating regions for the one or more extracted regions of interest.

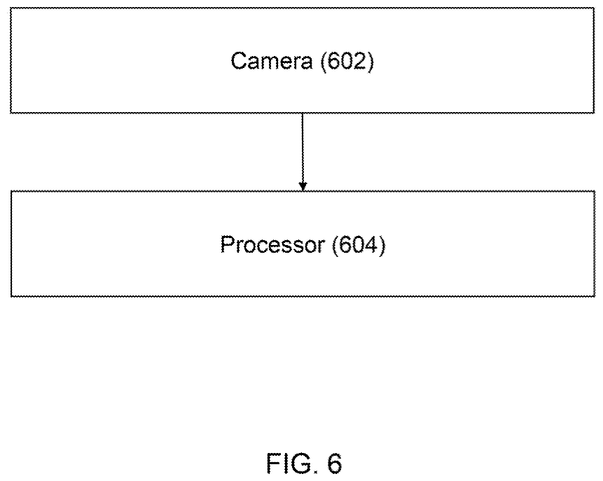

FIG. 6 illustrates exemplary physical components of an apparatus or a device for non-contact and non-invasive monitoring of blood sugar level of a user, in accordance with an exemplary embodiment of the present disclosure.

In an exemplary embodiment, an apparatus for non-contact and non-invasive monitoring of blood sugar level of a user is disclosed. The apparatus includes a camera (602) to obtain a real-time video of the user. The apparatus also includes a processor (604) of a system (not shown) coupled to the camera (602). The processor is configured to process at least each frame from the obtained real-time video; extract one or more facial regions from the each of the processed frames to thereby extract one or more regions of interest present therein; and feed the one or more extracted regions of interest to at least one image based physiological monitoring model along with one or more Photo plethysmography imaging (iPPG) and Optical Coherence Tomography(OCT) variations to process the one or more extracted regions of interest and obtain at least one result indicative of the blood sugar level of the of user based on the real-time video.

In an exemplary embodiment, the at least one image based physiological monitoring model utilizes an artificial intelligence (AI) or deep learning techniques or comprise of Convolutional Neural Network (CNN) algorithm or a software to obtain the at least one result.

In another exemplary embodiment, a device for non-contact and non-invasive monitoring of blood sugar level of a user. This includes a camera (602) to obtain a real-time video of the user. The apparatus also includes a processor (604) coupled to the camera (602). The processor is configured to process at least each frame from the obtained real-time video;

extract one or more facial regions from the each of the processed frames to thereby extract one or more regions of interest present therein; and feed the one or more extracted regions of interest to at least one image based physiological monitoring model along with one or more Photo plethysmography imaging (iPPG) and Optical Coherence Tomography(OCT) variations to process the one or more extracted regions of interest and obtain at least one result indicative of the blood sugar level of the of user based on the real-time video.

In an exemplary embodiment, the at least one image based physiological monitoring model utilizes an artificial intelligence (AI) or deep learning techniques or comprise of a computer algorithm or a software to obtain the at least one result.

In an exemplary embodiment, the processor is further configured to display the at least one obtained result on a user interface of the device, wherein the user interface displays the blood sugar level indication in at least one of a healthy range, a caution range, and an abnormal range category and an indication of one or more possible predicted diseases based on the at least one obtained result.

The present invention provides easy ways to use single location to make appointments across multiple organizations and multiple locations and types of medical tests and vaccinations. This is accessible via an app or website.

In an exemplary embodiment, FIG. 6 is a schematic structural diagram of Embodiment of apparatus or device according to the present invention. As shown in FIG. 6, apparatus or device provided by this embodiment includes a processor and a camera. The apparatus or device may further include a transmitter (not shown) and a receiver (not shown) and a memory (not shown). The memory, transmitter, and receiver are connected to the processor by using a bus. The memory stores an execution instruction; when the apparatus or device runs, the processor communicates with the memory; and the processor invokes the execution instruction in the memory to execute the operations as discussed in FIG. 5 above.

In an exemplary embodiment, the apparatus or a device or a server can be implemented in the computer system to enable aspects of the present disclosure. Embodiments of the present disclosure include various steps, which have been described above. A variety of these steps may be performed by hardware components or may be tangibly embodied on a computer-readable storage medium in the form of machine-executable instructions, which may be used to cause a general-purpose or special-purpose processor programmed with instructions to perform these steps. Alternatively, the steps may be performed by a combination of hardware, software, and/or firmware.

The computer system or a computing device or a server includes an external storage device, a bus, a main memory, a read only memory, a mass storage device, communication port, and a processor. A person skilled in the art will appreciate that computer system or a computing device or a server may include more than one processor and communication ports. Examples of processor 570 include, but are not limited to, an Intel® Itanium® or Itanium 2 processor(s), or AMD® Opteron® or Athlon MP® processor(s), Motorola® lines of processors, FortiSOC™ system on a chip processors or other future processors. Processor may include various modules associated with embodiments of the present invention. Communication port can be any of an RS-232 port for use with a modem based dialup connection, a 10/100 Ethernet port, a Gigabit or 10 Gigabit port using copper or fiber, a serial port, a parallel port, or other existing or future ports. Communication port may be chosen depending on a network, such a Local Area Network (LAN), Wide Area Network (WAN), or any network to which computer system connects. Memory can be Random Access Memory (RAM), or any other dynamic storage device commonly known in the art. Read only memory can be any static storage device(s) e.g., but not limited to, a Programmable Read Only Memory (PROM) chips for storing static information e.g., start-up or BIOS instructions for processor. Mass storage may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc. Bus communicatively couples processor(s) with the other memory, storage and communication blocks. Bus can be, e.g. a Peripheral Component Interconnect (PCI)/PCI Extended (PCI-X) bus, Small Computer System Interface (SCSI), USB or the like, for connecting expansion cards, drives and other subsystems as well as other buses, such a front side bus (FSB), which connects processor to software system. Optionally, operator and administrative interfaces, e.g. a display, keyboard, and a cursor control device, may also be coupled to bus to support direct operator interaction with computer system. Other operator and administrative interfaces can be provided through network connections connected through communication port. External storage device can be any kind of external hard-drives, floppy drives, IOMEGA® Zip Drives, Compact Disc-Read Only Memory (CD-ROM), Compact Disc-Re-Writable (CD-RW), Digital Video Disk-Read Only Memory (DVD-ROM). Components described above are meant only to exemplify various possibilities. In no way should the aforementioned exemplary computer system limit the scope of the present disclosure.

Although the proposed system has been elaborated as above to include all the main modules, it is completely possible that actual implementations may include only a part of the proposed modules or a combination of those or a division of those into sub-modules in various combinations across multiple devices that can be operatively coupled with each other, including in the cloud. Further the modules can be configured in any sequence to achieve objectives elaborated. Also, it can be appreciated that proposed system can be configured in a computing device or across a plurality of computing devices operatively connected with each other, wherein the computing devices can be any of a computer, a laptop, a smartphone, an Internet enabled mobile device and the like. All such modifications and embodiments are completely within the scope of the present disclosure.

As used herein, and unless the context dictates otherwise, the term “coupled to” is intended to include both direct coupling (in which two elements that are coupled to each other or in contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices are able to exchange data with each other over the network, possibly via one or more intermediary device.

Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

While some embodiments of the present disclosure have been illustrated and described, those are completely exemplary in nature. The disclosure is not limited to the embodiments as elaborated herein only and it would be apparent to those skilled in the art that numerous modifications besides those already described are possible without departing from the inventive concepts herein. All such modifications, changes, variations, substitutions, and equivalents are completely within the scope of the present disclosure. The inventive subject matter, therefore, is not to be restricted except in the protection scope of the appended claims.

10: subject/user

100: a local electronic imaging device

110: a local display

120: camera

122: an imaging lens

124: an image sensor array

126: a spectral filter

134: semi-transparent mirror

136: mirror

140: an ambient light detector

142: a motion detector

144: detectors

300: blood sugar level monitoring system

320: image processing electronics

330: a system controller

340: computer

345: memory or data storage

355: communication controller

360: network

410: wellness parameters

570: processor

602: camera

604: processor

What is claimed is:

1. A non-contact and non-invasive method for monitoring of blood sugar level of a user, the non-contact and non-invasive method comprising:
obtaining, by one or more cameras, a real-time video of the user;
processing, by a processor, at least each frame from the obtained real-time video;
extracting, by the processor, one or more facial regions from the each of the processed frames to thereby extract one or more regions of interest present therein;
feeding, by the processor, the one or more extracted regions of interest to at least one image based physiological monitoring model along with one or more Photoplethysmography imaging (iPPG) and Optical Coherence Tomography (OCT) variations to process the one or more extracted regions of interest and obtain at least one result indicative of the blood sugar level of the of user based on the real-time video by using Convolutional Neural Network algorithm.

2. The non-contact and non-invasive method of claim 1, wherein the at least one obtained result provides the blood sugar level indication in at least one of a healthy range, a caution range, and an abnormal range category.

3. The non-contact and non-invasive method of claim 1, wherein the at least one obtained result provides an indication of one or more possible predicted diseases based on the at least one obtained result.

4. The non-contact and non-invasive method of claim 1, wherein at least one image based physiological monitoring model.

5. The non-contact and non-invasive method of claim 1, wherein the one or more Photoplethysmography imaging (iPPG) and Optical Coherence Tomography (OCT) variations are feed to the at least one image based physiological monitoring model to add one or more corelation labels while obtaining the at least one result by using Convolutional Neural Network algorithm.

6. The non-contact and non-invasive method of claim 1, wherein the at least one image based physiological monitoring model utilizes an artificial intelligence (AI) or deep learning techniques or a trained classifier to obtain the at least one result.

7. The non-contact and non-invasive method of claim 1, wherein the at least one image based physiological monitoring model comprise of Convolutional Neural Network algorithm or a software to obtain the at least one result.

8. The non-contact and non-invasive method of claim 1, wherein the step of processing further comprising de-noising profiles and executing one or more augmentation on the denoised profiles.

9. The non-contact and non-invasive method of claim 1, wherein the step of extracting further comprising filtering the one or more regions of interest before providing to at least one image based physiological monitoring model.

10. The non-contact and non-invasive method of claim 1, wherein the step of processing the one or more extracted regions of interest by the at least one image based physiological monitoring model further comprising extracting multi corelating regions for the one or more extracted regions of interest.

11. An apparatus for non-contact and non-invasive monitoring of blood sugar level of a user, the apparatus comprising:
a camera to obtain a real-time video of the user;
a processor of a system coupled to the camera, the processor configured to:
process at least each frame from the obtained real-time video;
extract one or more facial regions from the each of the processed frames to thereby extract one or more regions of interest present therein; and
feed the one or more extracted regions of interest to at least one image based physiological monitoring model along with one or more Photoplethysmography imaging (iPPG) and Optical Coherence Tomography (OCT) variations to process the one or more extracted regions of interest and obtain at least one result indicative of the blood sugar level of the of user based on the real-time video.

12. The apparatus of claim 11, wherein the at least one image based physiological monitoring model utilizes an artificial intelligence (AI) or deep learning techniques or comprise of Convolutional Neural Network algorithm or a software to obtain the at least one result.

13. An device for non-contact and non-invasive monitoring of blood sugar level of a user, the apparatus comprising:
a camera to obtain a real-time video of the user;
a processor coupled to the camera, the processor configured to:
process at least each frame from the obtained real-time video;
extract one or more facial regions from the each of the processed frames to thereby extract one or more regions of interest present therein; and
feed the one or more extracted regions of interest to at least one image based physiological monitoring model along with one or more Photoplethysmography imaging (iPPG) and Optical Coherence Tomography (OCT) variations to process the one or more extracted regions of interest and obtain at least one result indicative of the blood sugar level of the of user based on the real-time video.

14. The device of claim 13, wherein the at least one image based physiological monitoring model utilizes an artificial intelligence (AI) or deep learning techniques or comprise of Convolutional Neural Network algorithm or a software to obtain the at least one result.

15. The device of claim 13, wherein the processor is further configured to display the at least one obtained result on a user interface of the device, wherein the user interface displays the blood sugar level indication in at least one of a healthy range, a caution range, and an abnormal range category and an indication of one or more possible predicted diseases based on the at least one obtained result.
